(19) United States
(12) Patent Application Publication
(10) Pub. No.: US 2011/0257107 A1
(43) Pub. Date: Oct. 20, 2011

(54) Compounds for Delivering Amino Acids or Peptides with Antioxidant Activity into Mitochondria

(75) Inventors: Shey-Shing Sheu, Pittsford, NY (US); Marion W. Anders, Pittsford, NY (US); Lin Xu, Lexington, MA (US); Virendra K. Sharma, Pittsford, NY (US); Dhanajaya Nauduri, Galveston, TX (US)

(73) Assignee: University of Rochester, Rochester, NY (US)

(21) Appl. No.: 13/169,604

(22) Filed: Jun. 27, 2011

Related U.S. Application Data
(63) Continuation of application No. 12/097,948, filed on Jul. 13, 2009, now abandoned.

(51) Int. Cl. A61K 38/06; A61K 31/164; C12N 5/00; A61P 39/06; C07C 323/59; C07D 277/06; A61K 31/223; A61K 31/426; C07C 323/60; C07K 5/083

(52) U.S. Cl. 514/21.9; 564/154; 514/616; 435/375; 530/331; 560/147; 548/201; 514/550; 514/365

(57) ABSTRACT

Disclosed are compounds containing single amino acids, peptides, or derivatives thereof which are selectively delivered to the mitochondria of a cell. Compounds of the invention exhibit antioxidant activity thereby reducing reactive oxygen species in cells. These compounds are useful for inhibiting oxidative stress-induced cell injury or death both in vivo and ex vivo. In addition, methods for the synthesis of these compounds are disclosed.

### INTRODUCTION

This application is a continuation of U.S. Ser. No. 12/097,948 filed Jul. 13, 2009 which is the National Phase application of PCT/US2006/062231 filed Dec. 18, 2006, which is based on U.S. Ser. No. 11/312,873 filed Dec. 20, 2005, the contents of which are incorporated herein by reference in their entirety.

### BACKGROUND OF THE INVENTION

Mitochondria occupy a central role in cellular homeostasis, particularly by satisfying cellular energy needs, and, paradoxically, also occupy a central role in a range of disease processes. Mitochondria are the major source (>90%) of adenosine triphosphate (“ATP”), which is used in a range of energy-requiring biochemical and homeostatic reactions in the body. Mitochondria are also a major source of reactive oxygen species (“ROS”), which are involved in the etiology and progression of a range of disease processes, including, for example, inflammation, stroke, cardiovascular disease, cancer, diabetes, neurodegenerative diseases (e.g., Alzheimer's Disease, Parkinson's Disease), drug-and chemical-induced toxicity, alcohol-induced liver damage, and aging-related diseases.

Antioxidant mechanisms in the body counteract the deleterious effects of ROS. These antioxidant mechanisms may, however, be overwhelmed during the development and progression of disease processes. The hydrophilic tripeptide glutathione (L-γ-glutamyl-L-cysteinylglycine) is an important antioxidant compound.

Unlike lipophilic antioxidants, which must be provided by the diet, glutathione is synthesized in the body, particularly in the liver. Glutathione is present in mitochondria, but mitochondria lack the enzymes needed for the synthesis of glutathione (Griffith and Meister (1985) Proc. Natl. Acad. Sci. USA 82:4668-4672), and the mitochondrial glutathione pool is maintained by transport from the cytosol into the mitochondria. The mitochondrial glutathione pool amounts to approximately 15% of total cellular glutathione (Meredith and Reed (1982) J. Biol. Chem. 257:3747-3753).

Although the mitochondrial glutathione pool is relatively small, it plays a key role in cytoprotection against ROS, and the depletion of mitochondrial glutathione concentrations is associated with cell damage and death (Meredith and Reed (1982) Biochem. Pharmacol. 32:1383-1388; Shan, et al. (1993) Chem. Res. Toxicol. 6:75-81; Hashmi, et al. (1996) Chem. Res. Toxicol. 9:361-364). In particular, depletion of mitochondrial glutathione concentrations sensitizes organs to cytokine (TNF)-associated cell damage (Colell, et al. (1998) Alcohol Clin. Exp. Res. 22:763-765; Colell, et al. (1998) Gastroenterology 115:1541-1551). The antioxidant activity of glutathione is associated with its thiol group.

### SUMMARY OF THE INVENTION

The present invention is an amino acid-based antioxidant compound selectively delivered into the mitochondria of a cell. In particular embodiments, the antioxidant compound of the invention is in admixture with a pharmaceutically acceptable carrier. Compounds of the present invention are produced by linking an amino acid-based antioxidant to a delivery moiety which selectively delivers the antioxidant into the mitochondria of a cell.

The present invention also embraces a method of inhibiting oxidative stress-induced cell injury or death by contacting a cell with a compound of the invention, whereby the compound is taken up by the cell and is selectively delivered into the mitochondria of the cell, thereby scavenging oxidative free radicals or reactive oxygen species to inhibit oxidative stress-induced cell injury or death.

The present invention is also a method of treating a condition associated with oxidative stress-induced cell injury or death. The method involves administering an effective amount of a pharmaceutical composition containing an antioxidant compound of the invention to a patient having a condition associated with oxidative stress-induced cell injury or death, whereby the compound is taken up by cells at risk of oxidative stress-induced injury or death, and is selectively transported into the mitochondria of the cells to inhibit oxidative stress-induced injury or death thereof, thereby treating the condition.

### BRIEF DESCRIPTION OF THE DRAWINGS

FIG. 1 shows a quantification of TMRE-fluorescence (ΔF/Fₒ) as a function of time after exposure to 3 μM and 3 mM concentrations of H₂0₂. The signals are from two different neuronal soma (N1, N2) and four neuritis (n1-n4). Of note is the considerable heterogeneity of response.

FIG. 2 demonstrates the ability of cysteine choline ester (CYS CE), N-acetyl cysteine choline ester (NAC CE), glutathione choline ester (Mito GSH), and N,S-acetyl-L-cysteine choline ester (Mito NAC) to minimize the depolarization of mitochondrial membrane potential induced by oxidative stress.

FIG. 3 demonstrates the ability of glutathione choline ester (Mito GSH) to delay the onset of H₂0₂-induced depolarization of mitochondrial membrane potential in cultured neonatal rat ventricular myocytes as compared to glutathione which is not selectively delivered to mitochondria.

FIG. 4 graphically represents the latency of H₂0₂-induced depolarization of mitochondrial membrane potential in control (H₂0₂), glutathione (GSH), and glutathione choline ester (Mito GSH), demonstrating the ability of Mito GSH to delay the onset of H₂0₂-induced depolarization of cultured neonatal rat ventricular myocytes.

FIG. 5 demonstrates the ability of N-acetyl-L-cysteine choline ester (mito NAC) to delay the onset of H₂0₂-induced depolarization of mitochondrial membrane potential in cultured neonatal rat ventricular myocytes.

FIG. 6 demonstrates that glutathione choline ester (Mito GSH) protects against N-methyl-D-aspartate (NMDA)-induced reactive oxygen species generation in brain striatal neurons.

### DETAILED DESCRIPTION OF THE INVENTION

The primary native mitochondrial mechanisms for counteracting the deleterious effects of ROS involve glutathione and derivatives thereof. Since mitochondria do not have the enzymes necessary for the synthesis of glutathione, the mitochondrial glutathione pool must be maintained. It has now been found that the characteristics of active mitochondrial transport systems and of the mitochondrial electrochemical potential gradient can be exploited to concentrate glutathione derivatives and other modified amino acid-based antioxidants in mitochondria, thereby providing critical mitochondrial antioxidant potential to counteract the effect of ROS.

Thus, the present invention embraces a compound composed of an amino acid-based antioxidant moiety linked to a delivery moiety, which facilitates the selective delivery of the antioxidant to the mitochondria of a cell. As used in the context of the present invention, “selectively delivered” or “selective delivery” is intended to mean that an amino acid-based antioxidant is modified in such a manner to produce a compound that is specifically transported across mitochondrial membranes by active mitochondrial transport systems such as the well-known choline transporters (Apparsundaram, et al. (2000) Biochem. Biophys. Res. Commun. 276:862-867; Okuda, et al. (2000) Nat. Neurosci. 3:120-125; Porter, et al. (1992) Biol. Chem. 267:14637-14646), carnitine acetyltransferase or dicarboxylate transporter; or the mitochondrial electrochemical potential gradient so that the antioxidant moiety accumulates in the mitochondria.

The compounds of the present invention are intended to provide antioxidant activity capable of preventing the formation of (or detoxify) free radicals, and/or to scavenge reactive oxygen species (e.g., superoxide, hydrogen peroxide, hypochlorous acid, ozone, singlet oxygen, hydroxyl radical, and peroxyl, alkoxyl, and hydroperoxyl radicals) or their precursors.

Antioxidant activity of the instant compound is provided by an amino acid-based antioxidant moiety, i.e., any individual amino acid or amino acid derivative that possesses such antioxidant activity. As used in the context of the present invention, an amino acid is intended to include amino acids that are relevant to the production of proteins as well as non-protein associated amino acids. Exemplary amino acids and derivatives thereof include, without limitation, glutamic acid, cysteine, N-acetyl-cysteine, glycine, and 2,2-dialkylthiazolidine-4-carboxylic acid.

In a particular embodiment, an amino acid-based antioxidant is composed of two or more amino acids or amino acid derivatives, defined herein as a peptide-based antioxidant moiety, wherein at least one or more of the amino acids or amino acid derivatives of the peptide possess antioxidant activity. Thus, in one embodiment, the peptide-based antioxidant moiety is at least two amino acids (or amino acid derivatives) in length, wherein at least one of the amino acids possesses antioxidant activity. In other embodiments, the peptide-based antioxidant moiety is from two to about ten amino acids (or amino acid derivatives) in length, wherein one or more of the amino acids possess antioxidant activity. In still further embodiments, the peptide-based antioxidant moiety is from two to about five amino acids (or amino acid derivatives) in length, wherein one or more of the amino acids possess antioxidant activity. Exemplary peptide-based antioxidant moieties for use in accordance with the instant compounds include, without limitation, L-γ-glutamylcysteine, L-γ-glutamylglycine, L-cysteinylglycine, glutathione, N-acetyl glutathione, L-carnosine, L-carnitine, and acetyl-L-carnitine.

As will be appreciated by one of skill in the art, the amino acids and their derivatives that form the antioxidant moiety can be L-amino acids or derivatives thereof, D-amino acids or derivatives thereof, or combinations thereof (e.g., in a peptide-based antioxidant moiety).

In general, selective delivery of the amino acid-based or peptide-based antioxidant is achieved by linking (e.g., via a covalent linkage) the amino acid-based or peptide-based antioxidant with a delivery moiety which by virtue of recognition by the mitochondrial transport system or charge and polarity facilitates delivery and accumulation of the antioxidant in mitochondria. Accordingly, particular embodiments embrace a delivery moiety which is specifically transported by a protein of the mitochondrial transport system. In other embodiments, the delivery moiety is hydrophilic. In still other embodiments the delivery moiety is positively charged. Exemplary delivery moieties include, but are not limited to, choline esters; choline ethers; carnitine esters; N-heterocycle esters such as aliphatic N-heterocycles (e.g., N-cyclopentyl, N-cyclohexyl, etc.); and N-heterocycles containing a ring nitrogen that can be in a quaternary state including rings with the nitrogen double-bonded with the ring structure (e.g., pyridinyl, pyrimidinyl, quinolinyl, isoquinolinyl, imidazolyl, pyrazolyl, pirazinyl, etc.) and rings with the nitrogen only single-bonded within the ring structure (e.g., pyrrolyl, pyrrolidinyl, morpholinyl, piperidinyl, etc.) and amide analogs of choline esters and N-heterocycle esters. Other such N-heterocycles are well-known to one of skill in the art and can be found in Handbook of Chemistry and Physics, 63 ed., page C-35, et seq.

The linker between the amino acid-based or peptide-based antioxidant and the delivery moiety can be any linker molecule that does not interfere with the antioxidant activity of the amino acid-based or peptide-based antioxidant and does not interfere with the transport or polarity of the compound imparted by the presence of the delivery moiety. The linker desirably contains up to, and including, about 20 molecules in a direct chain (i.e., excluding molecules in any sidechains) that links together the amino acid-based or peptide-based antioxidant and the delivery moiety (e.g., quaternary nitrogen or heterocycle that contains therein the quaternary nitrogen). Exemplary linkers include, without limitation, —Z¹—Z²—, —Z—O—Z²—, —Z¹—S—Z²—, —Z¹—N(H)—Z²—, —Z¹—CO—N(H)—Z²—, or —Z¹—N(H)—CO—Z²— where Z¹ is a direct link, an aliphatic or non-aliphatic C1 to C10 hydrocarbon, a single, fused or multi-ring aromatic, or an aliphatic or non-aliphatic cyclic group; and where Z² is an aliphatic or non-aliphatic C1 to C10 hydrocarbon, a single, fused or multi-ring aromatic, or an aliphatic or non-aliphatic cyclic group.

As used to define the linker, the term “aliphatic or non-aliphatic C1 to C10 hydrocarbon” refers to both alkyl groups that contain a single carbon and up to about 10 carbons, as well as alkenyl groups and an alkynyl groups that contain two carbons and up to about 10 carbons, whether the carbons are present in a single chain or a branched chain. Exemplary aliphatic or non-aliphatic C1 to C10 hydrocarbon include, without limitation, methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene, 3-butynylene, etc.

As used to define the linker, the term “single, fused or multi-ring aromatic” refers to any combination of aromatic ring structures, whether or not the ring(s) contain hetero-atoms. Exemplary single, fused or multi-ring aromatics include, without limitation, phenyl, biphenyl, triphenyl, napthyl, phenanthryl, anthracyl, etc.

As used to define the linker, the term “aliphatic or non-aliphatic cyclic group” refers to any non-aromatic cyclic structure, whether or not the cyclic structure contains one or more hetero-atoms. Exemplary aliphatic or non-aliphatic cyclic groups include, without limitation, aliphatic hydrocarbon cyclic structures such as cyclopentyl, cyclohexyl, cycloheptyl, etc., and non-aromatic hydrocarbon cyclic structures such as cyclopentenyl, cyclohexenyl, cyclopentadienyl, cyclohexadienyl, etc. Exemplary aliphatic or non-aliphatic heterocyclic groups include, without limitation, aliphatic or non-aliphatic N-heterocycles (e.g., aza- and diaza-cycloalkyls such as aziridinyl, azetidinyl, diazatidinyl, pyrrolidinyl, piperidinyl, piperazinyl, and azocanyl, pyrrolyl, pyrazolyl, imidazolyl, pyridinyl, pyrimidinyl, pyrazinyl, pyridazinyl, triazinyl, tetrazinyl, pyrrolizinyl, indolyl, quinolinyl, isoquinolinyl, benzimidazolyl, indazolyl, quinolizinyl, cinnolinyl, quinalolinyl, phthalazinyl, naphthyridinyl, quinoxalinyl, etc.), aliphatic or non-aliphatic S-heterocycles (e.g., thiranyl, thietanyl, tetrahydrothiophenyl, dithiolanyl, tetrahydrothiopyranyl, thiophenyl, thiepinyl, thianaphthenyl, etc.), and mixed heterocycles such as morpholinyl, thioxanyl, thiazolyl, isothiazolyl, thiadiazolyl, etc.

Particularly suitable compounds of the present invention include, without limitation, the following: carnitine and choline esters of N-acetyl glutathione, L-γ-glutamyl-L-cysteinylglycine choline ester, D-γ-glutamyl-L-cysteinylglycine choline ester, L-cysteine choline ester, L-γ-glutamyl-L-cysteine choline ester, D-γ-glutamyl-L-cysteine choline ester, N-acetyl-L-cysteine choline ester, N-acetyl-L-cysteine choline amide, glutathione choline ester, glutathione choline amide, D-2-(trimethylamino)ethyl-2,2-dimethylthiazolidine-4-carboxylic acid, and L-2-(trimethylamino)ethyl-2,2-dimethyl-thiazolidine-4-carboxylic acid, [2-(2-acetylamino-3-mercaptopropionyloxy)ethyl]trimethylammonium bromide, [2-(2)-amino-3-mercaptopropionyloxy)ethyl]trimethylammonium iodide, (2-{2-[2-(4-amino-4-carboxybutyrylamino)-3-mercapto-propionylamino]acetoxylethyl)trimethylammonium bromide, 2-amino-3-mercaptopropionic acid 2,2-dimethylaminoethyl ester.

According to one embodiment, the compound of the present can be any compound possessing an amino acid-based or peptide-based antioxidant linked to a delivery moiety, except that the compound is not glycine choline ester.

The compounds of the present invention can also be in the form of a salt, preferably a pharmaceutically acceptable salt. The term “pharmaceutically acceptable salt” refers to those salts that retain the biological effectiveness and properties of the free bases or free acids, and which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

The above-identified compounds, or their salts, can be prepared according to various procedures using different starting materials and reactants, as disclosed herein by way of example.

Having prepared the compounds of the present invention, such compounds can be used in forming a pharmaceutical composition that is intended for therapeutic uses of the type described hereinafter. Typically, the pharmaceutical composition of the present invention will include at least one compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions. In particular embodiments, the pharmaceutical composition employs a combination of the compounds of the present invention.

Typically, the composition will contain from about 0.01 to 99 percent, preferably from about 20 to 75 percent of active compound(s), together with the adjuvants, carriers and/or excipients. For example, application to mucous membranes and/or lungs can be achieved with an aerosol or nebulized spray containing small particles of a compound of this invention in a spray or dry powder form.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained.

Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy us of a syringe exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils.

Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container or metered dose inhaler together with suitable propellants, for example, hydrocarbon propellants like propane, butane, or isobutane with conventional adjuvants. The materials of the present invention also may be administered in a non-pressurized form such as in a nebulizer or atomizer.

As disclosed herein, an amino acid-based or peptide-based antioxidant compound of the present invention is readily taken up by cells and selectively delivered into the mitochondria inside the cells where the compounds can exert their effect as antioxidants, reducing the reactive oxygen species (ROS) that are generated in mitochondria following ROS-inducing events thereby affording cytoprotection to cultured cells and cells in vivo exposed to oxidative stress. In particular, the compounds of the present invention are useful to reduce ROS that occur following trauma or other events capable of inducing apoptosis, including excitotoxic apoptosis.

Therefore, the present invention also embraces a method of inhibiting oxidative stress-induced injury and/or death of a cell. Basically, a cell, whether located in vitro or in vivo, is contacted with the compound or its salt (as well as a pharmaceutical composition of the present invention), whereby the compound, presumably by virtue of its charged quaternary nitrogen or recognition by the mitochondrial transport system, is taken up by the cell and enters mitochondria of the cell. As a result of its entry in the cell and accumulation within the mitochondria, the amino acid-based or peptide based antioxidant moiety carried by the compound is able to exert its antioxidant activity within the mitochondrial environment, scavenging oxidative free radicals and/or reactive oxygen species to inhibit oxidative stress-induced injury and/or death. The cells to be treated in accordance with this aspect of the present invention can be any cell that possesses mitochondria, but desirably those mitochondria-containing cells that have a significant population of mitochondria therein. Exemplary cells include, without limitation, neuronal cells, muscle cells (e.g., skeletal or cardiac muscle cells), liver cells, and kidney cells.

By virtue of the ability to inhibit oxidative stress-induced injury and/or death of a cell, the present invention also affords a method of treating or preventing a condition associated with oxidative stress-induced injury and/or death. This aspect of the invention is carried out by administering a compound of the present invention, or its salt (as well as pharmaceutical compositions containing the same) to a patient having a condition associated with oxidative stress-induced cellular injury and/or death. As a result of such administration, the compound is readily taken up by cells at risk of oxidative stress-induced injury and/or death, and enters the mitochondria of such cells. As noted above, entry of the compound into cells and accumulation within the mitochondria allows the amino acid-based or peptide based antioxidant moiety carried by the compound to exert its antioxidant activity within the mitochondrial environment, scavenging oxidative free radicals and/or reactive oxygen species to inhibit oxidative stress-induced injury and/or death.

Administration of the compound of the invention (or pharmaceutical composition) can be carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, transmucosally, or via inhalation. Frequently, it will be necessary to repeat administration of the compound or pharmaceutical composition over a time course of several hours, or several days, weeks, or months. If the condition is a chronic condition, then administration may be carried out for an indeterminate period of time.

Conventional administration methods may be suitable for use in the present invention as described below.

Compounds or compositions within the scope of this invention include all compounds or compositions, wherein the compound of the present invention is contained in an amount effective to achieve its intended purpose. While individual needs vary, determination of optimal ranges of effective amounts of each component is within the skill of the art. The quantity of the compound or composition administered will vary depending on the patient and the mode of administration and can be any effective amount. Typical dosages include about 0.01 to about 100 mg/kg-body weight. The preferred dosages include about 0.01 to about 0.1 mg/kg-body weight up to three times a day. Treatment regimen for the administration of the compounds of the present invention can also be determined readily by those with ordinary skill in art. The quantity of the compound administered may vary over a wide range to provide in a unit dosage an effective amount of from about 0.01 to 20 mg/kg of body weight of the patient per day to achieve the desired effect.

Conditions to be treated or prevented in accordance with this aspect of the present invention are any condition, disease, disorder, or dysfunction that implicates ROS in the etiology of the condition, disease, disorder, or dysfunction. Exemplary conditions, diseases, disorders, and dysfunctions include, without limitation, stroke, neurodegenerative diseases (such as Alzheimer's Disease, Parkinson's Disease, Huntington's Disease, spinocerebellar ataxias), trauma (such as spinal cord injuries, skeletal or cardiac muscle injuries, kidney injuries, or liver injuries), muscular disorders (such as mitochondrial myopathy, lactic acidosis), diabetes, ischemia-reperfusion tissue injury, hypoxic-induced tissue damage, migraines, congenital mitochondrial diseases (such as MELAS, LHON, Kearns-Sayres Syndrome, MERRF, NARP, Leigh's Syndrome), neuromuscular degenerative disorders (such as Friedreich's Ataxia, Duchenne muscular dystrophy, Multiple Sclerosis), epilepsy, neuropathy, neurological and neuropsychological developmental delays, amyotrophic lateral sclerosis (Lou Gehrig's Disease), renal tubular acidosis, and aging related diseases or disorders (such as cognitive and motor disorders, progeria, cancer). While the above list is merely illustrative, a more complete list of mitochondrial diseases or disorders that can be treated in accordance with the present invention is provided in U.S. Pat. No. 6,472,378.

By treating, it is intended that the compounds and compositions of the invention can be used to diminish in whole or in part the symptoms associated with conditions, diseases, disorders, and dysfunctions that implicate mitochondrial oxidative stress. The administration of the compounds and compositions of the invention can, in certain circumstances, effectively minimize tissue damage associated with trauma or other events, or slow the progression of chronic diseases or dysfunctions.

The following examples are provided to illustrate embodiments of the present invention but are by no means intended to limit its scope.

### EXAMPLE 1

### General Synthesis of Choline/N-Heterocycle Esters of Amino Acid-Based or Protein-Based Antioxidants

According the disclosure herein, the compounds of the present invention can be represented by Formula I and Formula II:

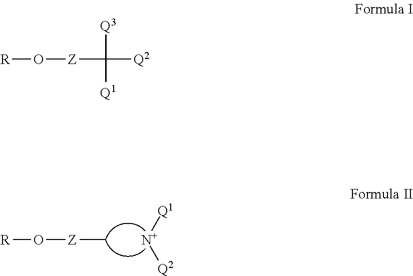

wherein R is the amino acid-based or peptide-based antioxidant moiety as disclosed herein; Z is the linker a described herein; and Q¹, Q², and Q³ are independently (i.e., independent of one another) aliphatic C1 to C5 hydrocarbons, such as methyl, ethyl, propyl, butyl, and pentyl groups or alternatively, for compounds of formula I, Q² and Q³ together form an aliphatic N-heterocycle; and wherein for Formula II, the N-heterocycle possesses a quaternary nitrogen and Q² is optional.

Compounds of Formula I and Formula II can be prepared using a variety of approaches. For example, in one approach, a final intermediate according to Formula III or Formula IV,

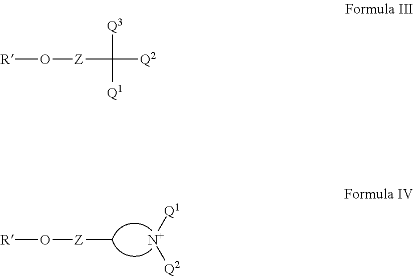

wherein R′ is a derivative of R having one or more protecting groups, is reacted with one or more agents that are effective to remove the one or more protecting groups, thereby forming the compound of Formula I or the compound of Formula II, respectively.

According to a more desirable approach, the intermediate according to Formula III or Formula IV is first exposed to trifluoroacetic acid under conditions effective to remove the one or more protecting groups (i.e., deprotect the intermediate), and subsequently exposed to a cation scavenger agent, such as triethyl silane, to form the compounds according to Formula I or Formula II. Removal of the protecting groups can be carried out under any suitable conditions known to those of skill in the art, but desirably using either trifluoroacetic acid in dichloromethane, hydrogen bromide or hydrogen chloride in acetic acid, or tri-n-butyl phosphine.

An intermediate of Formula III can be prepared according to any one of several exemplary approaches. In a first approach, an intermediate according to Formula V

R′—O—Z—Br Formula V

is reacted with Q¹-N(Q²)-Q³ under conditions effective to form the intermediate according to Formula III. As an alternative to the intermediate of Formula V, its homologs containing iodine or chlorine can also be used. Typically, this step is performed in THF at room temperature for a sufficient amount of time (i.e., overnight up to about 48 hours). The intermediate of Formula V and its homologs are prepared by reacting an intermediate according to Formula VI

R′—OH Formula VI

with HO—Z—Br (or HO—Z—I or HO—Z—Cl) under conditions effective to form the intermediate according to Formula V or its homologs. Exemplary conditions include the use of (i) DCC or diisopropylcarbodiimide (DIC) and 4-dimethylaminopyridine followed by (ii) dichloromethane at room temperature for about 6 to 24 hours, desirably about 12 hours.

In a second approach, the intermediate according to Formula III is prepared by reacting an intermediate according to Formula VII

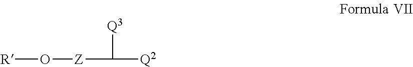

with IQ¹ under conditions effective to form the intermediate according to Formula III. Typically, this step is performed in ethyl acetate for a sufficient amount of time (i.e., overnight up to about 48 hours).

In a third approach, wherein the compound to be prepared is a protected glutathione choline ester, the synthesis can be carried out by reacting N-trimethyl-alkyl glycine ester with protected L-γ-glutamyl-L-cysteine under conditions effective to form the intermediate according to Formula III. This can be achieved according to the synthesis procedure described in Example 3, infra.

The intermediate according to Formula IV is prepared by reacting an intermediate according to Formula VIIIa or Formula VIIIb with

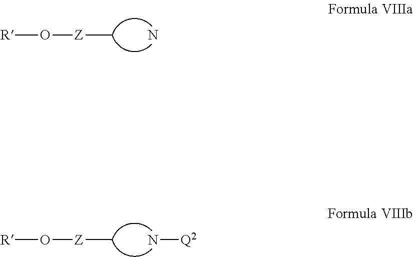

with I-Q¹ under conditions effective to form the intermediate according to Formula IV. Typically, this step is performed in ethyl acetate for a sufficient amount of time (i.e., overnight up to about 48 hours).

The intermediates according to Formula VII and Formula VIIIa or Formula VIIIb can be prepared by reacting an intermediate R′—OH with either HO—Z—N(Q²)-Q³ or HO—Z—(N-heterocyclic amine) or HO—Z—(N-heterocyclic amine)-Q² under conditions effective to form the intermediate according to Formula VII or Formula VIII, respectively. Exemplary conditions include the use of (i) DCC or DIC and 4-dimethylaminopyridine followed by (ii) dichloromethane at room temperature for about 6 to 24 hours, desirably about 12 hours.

For the compounds according to Formula I or Formula II where R is L-cysteine, the dimethylthiazolidine derivative thereof can be prepared by treating the compound(s) with acetone under effective conditions. Such a compound according to Formula I is (R)-2-(trimethylamino)ethyl-2,2-dimethylthiazolidine-4-carboxylic acid.

Amide analogs of choline esters, e.g., glutathione choline amide and N-acetyl-L-cysteine choline amide are formed in accordance with established methods by reacting an acid chloride, acid anhydride, or ester with the respective amines disclosed herein.

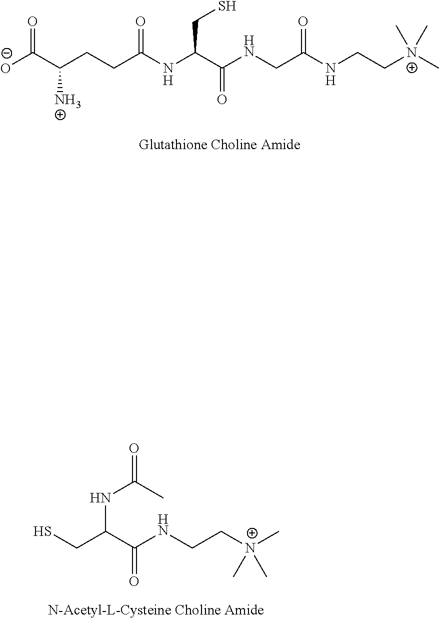

### EXAMPLE 2

### Synthesis of N-Acetyl L-Cysteine and L-Cysteine Choline Esters, and (R)-[2-(2,2-Dimethylthiazolidine-4-carbonyloxy)ethyl]trimethylammonium Chloride

All reactions were carried out under dry N₂ except where noted. All solvents were distilled from drying agents. Reagents were purchased from Aldrich and VWR. Normal phase column chromatography was performed on Silica Gel 60 (230-400 Mesh, EM Science). Reverse phase column chromatography was performed on BAKERBOND™ C₁₈ (40 μm, J. T. Baker). ¹H, ¹³C, and COSY NMR data were recorded on a Bruker AVANCE™ 400 with Me₄Si as the internal standard except where noted. MS analyses were performed with an Agilent LC/MSD ion-trap mass spectrometer (Agilent Technologies) with an electrospray interface operated in the positive-ion mode.

Scheme 1 shows an exemplary method for the synthesis of N-acetyl L-cysteine choline ester.

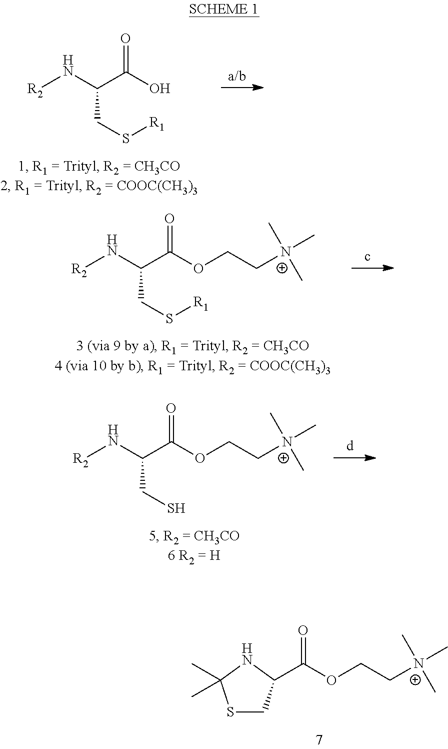

[2-(2-Acetylamino-3-tritylsulfanyl-L-propionyloxy)ethyl]trimethylammonium bromide (3). To a solution of 2-acetylamino-3-tritylasulfanyl-L-propionic acid (1) (2.5 g, 6.26 mmol), DCC (2.58 g, 12.5 mmol), 4-dimethylamino pyridine (1.53 g, 12.5 mmol), and 4-dimethylamino pyridinium chloride (1.99 g, 12.5 mmol) in CH₂Cl₂ (50 ml) was added 2-bromo-ethanol (1.33 ml, 18.8 mmol). After stirring for 12 hours at room temperature, the mixtures were filtered and extracted with 0.1% HCl, water, and brine subsequently. The extracted CH₂Cl₂ solution was dried with anhydrous MgS0₄ and evaporated to dryness. The white residue was purified by chromatography on silica gel (ethyl acetate/methanol 45:50) to give 2-acetylamino-3-tritylsulfanyl-L-propionic acid 2-bromoethyl ester (2.2 g, 70%) as a white solid; ¹HNMR (CDCl₃, 400 MHz): 7.40-7.38 (m, 6H), 7.29-7.18 (m, 9H), 6.07 (d, 1H, J=7.77 Hz), 4.55 (dd, 1H, J=6.35 and 4.59 Hz), 4.35 (t, 2H, J=6.94 Hz), 3.41 (t, 2H, J=6.94 Hz), 2.74 (dd, 1H, J=6.35 and 12.5 Hz), 2.61 (dd, 1H, J=4.59 and 12.5 Hz), 1.92 (s, 3H). ¹³C NMR (CDCl₃, 100 MHz): 169.9, 169.6, 144.1, 129.3, 127.9, 126.8, 66.9, 64.4, 51.1, 33.5, 28.0, 22.8; Electrospray-ion trap-MS: Calcd for C₂₆H₂₆BrNO₃S: m/z 511.1 & 513.1, Found: m/z 534.0 & 535.9 [M+Na]⁺.

At −78° C., liquid trimethylamine (1 ml, 10.5 mmol) was added to a solution of 2-acetylamino-3-tritylsulfanyl-L-propionic acid 2-bromoethyl ester (660 mg, 1.29 mmol) in THF (20 ml). The solution was allowed to warm to room temperature. After stirring for 48 hours, the formed white precipitate was filtered and rinsed with THF (5 ml×2) to give product 3 (600 mg, 81%). ¹H NMR (DMSO-d₆, 400 MHz): δ 7.34-7.22 (m, 15H), 4.40 (t, 2H, J=4.34 Hz), 4.12 (dd, 1H, J=10.9 & 12.2 Hz), 3.66 (t, 2H, J=4.34 Hz), 3.43 (d, 1H, J=3.43 Hz, NH), 3.10 (s, 9H), 2.63 (dd, 1H, J=10.9 & 4.54 Hz), 2.42 (dd, 1H, J=4.54 & 12.2 Hz), 1.85 (s, 3H); ¹³C NMR (DMSO-d₆, 100 MHz): δ 170.0, 169.8, 144.3, 129.3, 128.4, 127.2, 66.5, 63.6, 59.0, 53.0, 51.7, 32.6, 22.5; Electrospray-ion trap-MS: Calcd for C₂₉H₃₅N₂O₃S⁺: m/z 491.2. Found: m/z 491.2 [M]⁺.

N-acetyl-L-cysteine choline ester (5): To a solution of 3 (400 mg, 0.7 mmol) in CH₂Cl₂ (10 ml) was added to Et₃SiH (390 μl, 2.4 mmol) and anhydrous CF₃COOH (3 ml) subsequently. The mixtures were stirred at room temperature for 1 hour. The solution was dried under reduced pressure. The oily residue was dissolved into Et₂O (15 ml) and 1% HCl aqueous solution (15 ml). The aqueous solution was separated, rinsed twice with Et₂O (5 ml), neutralized by 10% NaHCO₃ to pH 7.0, and then lyophilized. The residue was purified by a preparative reversed-phase C18 column (20 cm×2.5 cm) with 5% CH₃CN in H₂O as eluent to give product 5 as a chloride salt (156 mg, 89%); ¹H NMR (DMSO-d₆/D₂0, 400 MHz): 6 4.56 (t, 1H, J=6.44 Hz), 4.45 (t, 2H, J=3.20 Hz), 3.60 (t, 2H, J=3.20 Hz), 3.10 (dd, 1H, J=6.40 & 10.0 Hz), 3.03 (s, 9H), 2.91 (dd, 1H, J=6.40 & 10.0 Hz), 1.87 (s, 3H); ¹³C NMR (DMSO-d₆, 100 MHz): δ 175.3, 172, 65.8, 60.9, 55.3, 53.4, 53.3, 23.2; Electrospray-ion trap-MS: Calcd for C₁₀H₂₁N₂0₃S⁺: m/z 249.1. Found: m/z 249.0 [M]⁺.

Scheme 1 also shows an exemplary method for the synthesis of L-cysteine choline ester.

[2-(2-tert-Butoxycarbonylamino-3-tritylsulfanyl-L-propionyloxy)ethyl]trimethylammonium iodide (4). To a solution of boc-L-Cys (trityl)-OH (2) (2 g, 4.3 mmol), DCC (1.78 g, 8.6 mmol), 4-dimethylaminopyridine (1.05 g, 8.6 mmol), and 4-dimethylaminopyridinium chloride (1.37 g, 8.6 mmol) in CH₂Cl₂ (50 ml) was added 2-(dimethylamino)ethanol (1.2 ml, 12 mmol). After stirring for 12 hours at room temperature, the mixtures were filtered and extracted with 0.1% HCl, water, and brine subsequently. The extracted CH₂Cl₂ solution was dried with anhydrous MgS0₄ and evaporated to dryness. The white residue was purified by chromatography on silica gel (ethyl acetate/methanol 65:35) to give 2-tert-butoxycarbonylamino-3-tritylsulfanyl-L-propionic acid 2-dimethylaminoethyl ester (1.6 g, 67%) as a white solid; ¹H NMR (CDCl₃, 400 MHz): 7.40-7.38 (m, 6H), 7.25-7.15 (m, 9H), 5.32 (d, 1H, J=8.24 Hz), 4.30 (dd, 1H, J=8.24 & 5.27 Hz), 4.17 (t, 2H, J=5.77 Hz), 2.60 (d, 2H, J=5.27 Hz), 2.49 (t, 2H, J=5.78 Hz), 2.19 (s, 6H), 1.42 (s, 9H); ¹³C NMR (CDCl₃, 100 MHz): 170.5, 154.7, 144.0, 129.2, 127.7, 126.5, 79.5, 66.4, 63.1, 57.1, 52.2, 45.4, 33.9, 28.0; Electrospray-ion trap-MS: Calcd for C₃₁H₃₈N₂0₄S: m/z 534.3, Found: m/z 535.0 [M+H]⁺& 557.1 [M+Na]⁺.

To a solution of 2-tert-butoxycarbonylamino-3-tritylsulfanyl-L-propionic acid 2-dimethylaminoethyl ester (1.5 g, 2.8 mmol) in THF (20 ml) was added methyl iodide (0.87 ml, 14 mmol). After stirring for 12 hours at room temperature, the mixtures were filtered and rinsed with THF (5 ml×2) to give product 4 as a white solid (2.1 g, 90%); ¹H NMR (CDCl₃, 400 MHz): 7.37-7.20 (m, 15H), 5.09 (d, 1H, J=6.98 Hz, NH), 4.60 (dd, 1H, J=15.2 & 6.40 Hz), 4.47 (dd, 1H, J=15.0 & 4.44 Hz), 4.09 (t, 2H, J=6.63 Hz), 3.95 (dd, 2H, J=4.44 & 6.40 Hz), 3.40 (s, 9H), 2.64 (dd, 2H, J=6.63 Hz) 1.39 (s, 9H); ¹³C NMR (CDCl₃, 100 MHz): 170.7, 154.7, 143.7, 129.0, 127.8, 126.6, 79.8, 66.7, 64.4, 59.9, 58.5, 54.1, 32.8, 27.9; Electrospray-ion trap-MS: Calcd for C₃₂H₄₁N₂0₄S⁺: m/z 549.3. Found: m/z 549.1 [M]⁺.

[2-(2-Amino-3-mercapto-L-propionyloxy)ethyl]trimethylammonium chloride (6, L-cysteine choline ester chloride). To a solution of 4 (1.3 g, 1.93 mmol) in CH₂Cl₂ (20 ml) was added to Et₃SiH (2.28 ml, 14.3 mmol) and anhydrous CF₃COOH (6 ml) subsequently. The mixtures were stirred at room temperature for 1 hour. The solution was dried under reduced pressure. The residue was dissolved into Et₂O (25 ml) and 1% HCl aqueous solution (25 ml). The aqueous solution was separated, rinsed twice with Et₂O (5 ml×2), neutralized by 10% NaHCO₃ to pH 7.0, and then lyophilized. The residue was purified by a preparative reversed-phase C18 column (20 cm×2.5 cm) with 5% CH₃CN in H₂O as eluent to give product 6 as a chloride salt (398 mg, 85%); ¹H NMR (D₂0/CD₃0D, 400 MHz): 4.76 (br, 1H), 4.51 (t, 2H, J=5.00 Hz), 3.83 (t, 2H, J=4.91 Hz), 3.24 (s, 9H), 3.17 (d, 2H, J=4.58 Hz); ¹³C NMR (CDCl₃, 100 MHz): 168.3, 65.3, 61.1, 55.4, 54.7, 24.7; Electrospray-ion trap-MS: Calcd for C₈H₁₉N₂0₂S⁺: m/z 207.1. Found: m/z 207.0 [M]⁺.

Scheme 1 also shows an exemplary method for the synthesis of (R)-[2-(2,2-Dimethyl-thiazolidine-4-carbonyloxy)ethyl]trimethylammonium chloride.

(R)-[2-(2,2-Dimethylthiazolidine-4-carbonyloxy)ethyl]trimethylammonium chloride (7). Compound (150 mg, 0.62 mmol) was dissolved into 10 ml acetone. After 20 minutes, the precipitate was filtered and rinsed with acetone (5 ml×2) to give 7 as a white solid (161 mg, 92%); ¹H NMR (D₂O/CD₃0D, 400 MHz): 5.15 (t, 1H, J=8.31 Hz), 3.87 (m, 2H), 3.85 (m, 1H), 3.76 (dd, 1H, J=8.25 & 12.0 Hz), 3.64 (dd, 1H, J=8.31 & 12.0 Hz), 3.26 (s, 9H), 3.21 (dd, 1H, J=5.10 & 12.0 Hz), 1.86 (s, 3H), 1.84 (s, 3H). ¹³C NMR (D₂O/CD₃0D, 100 MHz): 167.4, 73.9, 65.3, 62.5, 61.4, 54.8, 32.2, 28.9, 27.6; Electrospray-ion trap-MS: Calcd for C₁₁H₂₃N₂O₂S⁺: m/z 247.1. Found: m/z 247.1 [M]⁺.

### EXAMPLE 3

### Synthesis of Glutathione Choline Ester

All reactions were carried out under dry N₂ except where noted. All solvents were distilled from drying agents. Reagents were purchased from Aldrich and VWR. Normal phase column chromatography was performed on Silica Gel 60 (230-400 Mesh, EM Science). Reverse phase column chromatography was performed on BAKERBOND™ C₁₈ (40 μm, J. T. Baker). ¹H, ¹³C, and COSY NMR data were recorded on a Bruker AVANCE™ 400 with Me₄Si as the internal standard except where noted. MS analyses were performed with an Agilent LC/MSD, ion-trap mass spectrometer (Agilent Technologies) with an electrospray interface operated in the positive-ion mode.

Scheme 2 shows exemplary approach for the synthesis of glutathione choline ester.

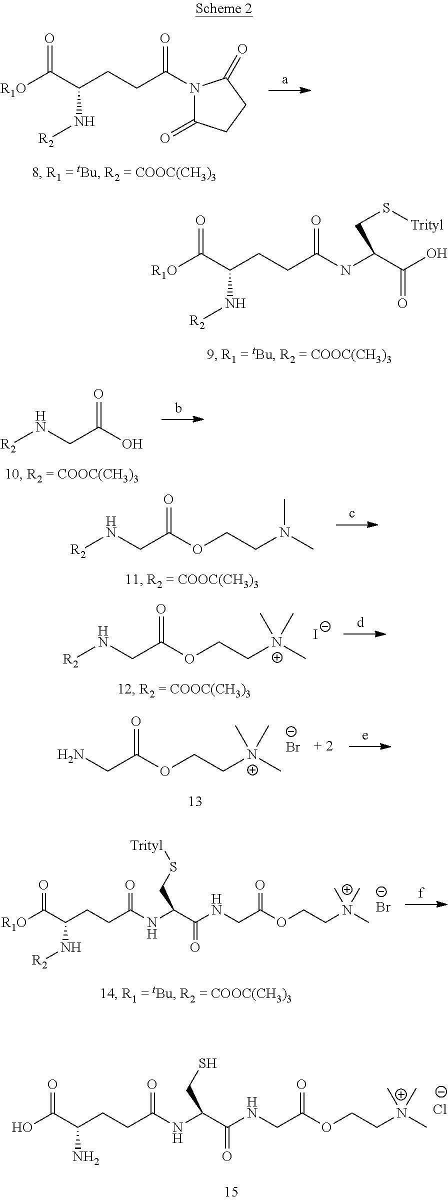

The α-carboxylic acid, and amino groups of glutamic acid were protected by forming tert-butyl (^{t}Bu) ester and tert-butyl carbamate, respectively. The thiol group of cysteine was protected as a trityl thioether. The key step in the synthesis was coupling of the protected L-γ-glutamyl-L-cysteine (Marsh, et al. (1997) Tetrahedron 53:17317-17334), and glycine choline ester (Mndzhoyan, et al. (1980) Khimiko-Farmatsevticheskii Zhurmal 14:34-36) to afford glutathione choline ester catalyzed by DIC (Flohr, et al. (1999) Chemistry 5:669-681). Simultaneous deprotection of ^{t}Bu, tert-butyloxycarbonyl, and trityl groups were accomplished by trifluoroacetic acid with a carbocation scavenger triethylsilane.

Boc-L-glutamyl-α-O-tert-butyl-τ-(S-trityl-L-cysteine) (9). To a solution of boc-L-glutamyl-α-tert-butyl-N-oxosuccinimide ester (Henderson, et al. (1999) J. Chem. Soc. Perkn. Trans. 1:911-914) (1.04 g, 2.6 mmol) in DMF (10 ml) was added S-trityl-L-cysteine (554 mg, 2.6 mmol) and triethylamine (0.362 ml, 2.5 mmol). The mixtures were stirred at room temperature for 12 hours. To the resulting mixture was added 20 ml 5% citric acid with extraction carried out using ethyl acetate (20 ml×3). The organic extract was rinsed with water (20 ml×2) and brine (20 ml×2) and dried with anhydrous MgSO₄. The dried EtOAc extract was filtered and filtrate was evaporated to give a crude residue. The residue was purified by chromatography on silica gel (ethyl acetate/hexane 1:1) to give 9 as a white solid (1.36 g, 81%); ¹H NMR (Acetone-d₆, 400 MHz): 7.42-7.22 (m, 15H), 6.30 (d, 1H, J=8 Hz, NH), 4.43 (dd, 1H, J=7.16 & 12.5 Hz), 4.05 (m, 1H), 2.68 (dd, 1H, J=7.49 & 12.2 Hz), 2.60 (dd, 1 H, J=5 & 12.2 Hz), 2.37 (t, 2H, J=7.41 Hz), 2.08 (m, 1H), 1.92 (m, 1H), 1.43 (s, 9H), 1.40 (s, 9H); ¹³C NMR (Acetone-d₆, 100 MHz): 172.6, 172.3, 172.0, 156.4, 145.4, 130.2, 128.7, 127.6, 81.4, 79.1, 67.2, 54.9, 52.1, 49.7, 34.4, 32.6, 28.5, 28.1; Electrospray-ion trap-MS: Calcd for C₃₆H₄₄N₂0₇S: m/z 648.3. Found: m/z 671.1 [M+Na]⁺.

Boc-glycine-2-(dimethylamino)ethyl ester (11). To a solution of boc-glycine (1.0 g, 5.7 mmol), DCC (1.82 g, 8.84 mmol), and triethylamine (0.84 ml, 6.05 mmol) in CH₂Cl₂ at 0° C. was added 2-(dimethylamino)-ethanol (1.5 ml, 14.6 mmol). The mixtures were allowed to warm to room temperature. After stirring for 12 hours, the solution was filtered, extracted with 1% HCl, saturated NaHCO₃, water, and brine subsequently. The extracted CH₂Cl₂ solution was dried with anhydrous MgS0₄ and evaporated to dryness. The white residue was purified by chromatography on silica gel (ethyl acetate/methanol 9:1) to give 11 (1.03 g, 74%) as a white solid; ¹H NMR (C₆D₆, 400 MHz): 5.51 (t, 1H, J=5.90 Hz, NH), 4.01 (t, 2H, J=5.88 Hz), 3.75 (d, 2H, J=5.90 Hz), 2.22 (t, 2H, J=5.88 Hz), 1.98 (s, 6H), 1.40 (s, 9H); ¹³C NMR (C₆D₆, 100 MHz): 170.4, 156.0, 79.1, 62.8, 57.7, 45.4, 42.7, 28.4; Electrospray-ion trap-MS: Calcd for C₁₁H₂₂N₂0₄: m/z 246.2. Found: m/z 247.0 [M+H]⁺.

Boc-glycine choline ester iodide (12). To a solution of boc-glycine-2-(dimethylamino)ethyl ester (1.2 g, 4.88. mmol) in THF at 0° C. was added methyl iodide (1.5 ml, 24.4 mmol). The solution was stirred for 12 hours at room temperature. The formed white precipitate was filtered to give 12 (1.76 g, 93%) as a white solid; ¹H NMR (CD₃OD, 400 MHz): 4.04 (b, 2H), 3.92 (s, 2H), 3.30 (b, 2H), 2.75 (s, 9H), 0.83 (s, 9H); ¹³C NMR (CD₃OD, 100 MHz): 171.4, 158.2, 80.8, 66.0, 59.8, 55.0, 43.5, 28.9; Electrospray-ion trap-MS: Calcd for C₁₂H₂₅N₂0₄⁺: m/z 261.2. Found: m/z 261.0 [M]⁺.

Glycine choline ester bromide (13). A solution of boc-glycine choline ester iodide (1.76 g, 4.5 mmol) in HBr in glacial acetic acid (30%, 8 ml) was stirred for 30 minutes at room temperature. After addition of ice-cold Et₂O (100 ml), the brown precipitate was filtered to give 13 as a yellowish solid; ¹H NMR (CD₃OD/D₂0, 400 MHz): 4.13 (t, 2H, J=4.63 Hz), 3.40 (s, 2H), 3.28 (t, 2H, J=4.63 Hz), 2.68 (s, 9H); ¹³C NMR (CD₃OD/D₂O, 100 MHz): 167.9, 65.7, 60.6, 54.9, 41.4; Electrospray-ion trap-MS: Calcd for free base C₇H₁₇N₂0₂⁺: m/z 161.1. Found: m/z 161.0 [M]⁺.

N-Boc-α-O-tert-butyl-τ-(S-trityl)glutathione choline ester bromide (14). A solution of 9 (1.43 g, 2.2 mmol), HOBt (311 mg, 2.3 mmol), and DIC (438 μl, 2.3 mmol) in CH₂Cl₂ (30 ml) was stirred for 30 min and then added a solution of give glycine choline ester bromide (0.7 g, 2.2 mmol) and triethylamine (0.322 ml, 2.3 mmol) in DMF (20 ml). After 24 hours, the brown solution was concentrated by evaporating CH₂Cl₂ under reduced pressure. The yellowish product was precipitated by addition of ice-cold Et₂O (50 ml). The solution was decanted and the precipitate was rinsed with Et₂O (10 ml×2). The precipitate was re-dissolved into CH₃CN and recrystallized in Et₂O to yield 14 (1.2 g, 64%) as a yellowish sold; ¹H NMR (Acetone-d₆, 400 MHz): 7.42-7.22 (m, 15H), 4.58 (br, 2H), 4.38 (br, 1H), 3.99 (br, 2H), 3.97 (br, 1H), 3.89 (br, 2H), 3.40 (s, 9H), 2.75 (m, 1H), 2.57 (m, 1H), 2.43 (m, 2H), 2.08 (m, 1H), 1.98 (m, 1H), 1.41 (s, 9H), 1.38 (s, 9H); ¹³C NMR (Acetone-d₆, 100 MHz): 172.7, 172.2, 171.4, 169.4, 156.2, 145.1, 129.9, 128.4, 127.1, 80.9, 78.7, 78.6, 66.8, 65.6, 64.8, 59.0, 54.8, 54.0, 53.1, 41.6, 34.4, 28.2, 27.7; Electrospray-ion trap-MS: Calcd for C₄₃H₅₉N₄0₈S⁺: m/z 791.4. Found: m/z 791.0 [M]⁺.

Glutathione choline ester chloride (15). To a solution of 14 (766 mg, 0.88 mmol) in CH₂Cl₂ (15 ml) was added to Et₃SiH (1.1 ml, 6.9 mmol) and anhydrous CF₃COOH (8 ml) subsequently. The mixtures were stirred at room temperature for approximately 3 hours until the color of solution did not change. The solution was dried under reduced pressure. The oily residue was dissolved into Et₂O (15 ml) and 1% HCl aqueous solution (15 ml). The aqueous solution was separated, rinsed twice with Et₂0 (5 ml), neutralized by 10% NaHCO₃ to pH 7.5, and then lyophilized to give the yellowish crude residue. The residue was purified by a preparative reversed-phase C18 column (20 cm×2.5 cm) with 1% CH₃CN in H₂O as eluent to give 15 (328 mg, 87%); ¹H NMR (CD₃OD/D₂O, 400 MHz): 4.63 (t, 2H, J=4.64 Hz), 4.55 (t, 1H, J=6.08 Hz), 4.09 (s, 2H), 3.88 (t, 1H, 6.40 Hz), 3.76 (t, 2H, J=4.64 Hz), 3.20 (s, 9H), 2.93 (m, 2H), 2.56 (m, 2H), 2.18 (m, 2H); ¹³C NMR (CD₃OD/D₂O, 100 MHz): 175.7, 174.6, 173.6, 171.1, 65.3, 60.0, 56.5, 54.7, 42.2, 32.1, 32.0, 27.0, 26.3; Electrospray-ion trap-MS: Calcd for C₁₅H₂₉N₄0₆S⁺: m/z 393.2. Found: m/z 393.2 [M]⁺.

### EXAMPLE 4

### Oxidative Stress in Spinal Cord Neurons

Central to the therapeutic intervention of spinal cord injury is the belief that spinal cord neurons undergo apoptosis and possibly necrosis under oxidative stress. To demonstrate that H₂0₂-induced changes are indicative of neuronal apoptosis in a cultured spinal cord neuron model system, spinal cord neurons were, treated with H₂0₂ and compared to non-treated spinal cord neurons. The results of this analysis showed condensed and fragmented nuclei in the H₂0₂-treated neurons indicative of apoptosis. In contrast, control neurons show predominantly diffuse nuclear staining. Quantification of these data demonstrated a significant increase in the number of neurons with condensed and fragmented nuclei after H₂0₂ treatment. At 250 μM H₂0₂, ˜42% of neurons exhibited condensed and fragmented nuclei as compared to the ˜6% observed in control neurons. This percentage increased to ˜58% for 500 μM H₂O₂. Selective depletion of mitochondrial glutathione by 3-hydroxy-4-pentenoate (3-HP) (Shan, et al. (1993) Chem. Res. Toxicol. 6:75-81; Hashmi, et al. (1996) Chem. Res. Toxicol. 9:361-364), prior to 250 μM H₂0₂ treatment increased percentage of cells showing these changes to 70%. These changes were concentration-dependent and increased by mitochondrial glutathione depletion. Immunohistochemical staining for cytochrome C showed punctate immunoreactivity in control neurons and a loss of this discrete localization in apoptotic cells, indicating that H₂0₂ induced cytochrome C release. In contrast, immunoreactivity to cytochrome C oxidase (COX), a marker in the inner mitochondrial membrane remained independent of the mitochondrial insults by H₂0₂. The differential staining between cytochrome C and COX affords a secondary method to assess whether a given neuron has undergone mitochondrial permeability transition (MPT).

### EXAMPLE 5

### H₂0₂ Induces Mitochondrial Permeability Transition

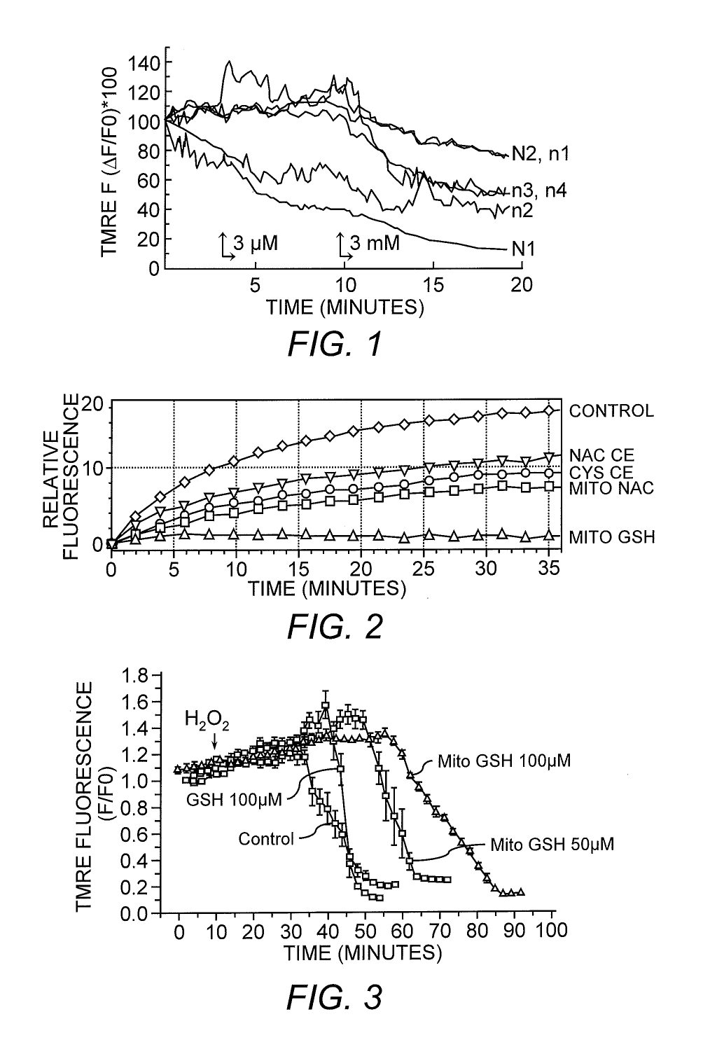

Cells treated with H₂0₂ also exhibited a time-dependent loss of mitochondrial TMRE fluorescence indicative of dissipated ΔΨₘ. This loss of punctate or filamentous fluorescence by H₂0₂ was not due to photobleaching because the control experiment, performed without H₂0₂, showed that the punctuate pattern after 20 minutes of recording was still preserved. Cyclosporin A (CsA) was found to inhibit the effect of H₂0₂, indicating that loss of TMRE fluorescence was an accurate measure of MPT. Further quantitative analysis of the cell images revealed two unexpected observations. First, mitochondria within a given neuronal soma behaved similarly. Second, significant heterogeneity in the timing of MPT existed depending on the subcellular location of the mitochondria within a neuron (e.g., soma vs. neurites) (FIG. 1).

### EXAMPLE 6

### Neuronal Glutathione Levels

Cellular and subcellular glutathione levels were determined with fluorescence microscopy using the glutathione-reactive fluorescent probe MC1B. This reporter is non-fluorescent in its native state but turns fluorescent when reacted with glutathione; the final conjugate exhibiting excitation in the UV range (excitation 385 nm, emission 485 nm). MC1B is well-known for its use in determining cellular glutathione levels (Fricker, et al. (2000) J. Microscopy 198:162-173; Tauskela, et al. (2000) Glia 30:329-341). Both phase bright neuronal and flat background glia cells were fluorescent indicating presence of glutathione in both cell types. Semi-permeablization of the neuronal membrane with saponin released cellular MC1B-glutathione conjugate leaving the mitochondrial fluorescence intact. This allowed assessment of mitochondrial glutathione in neurons in situ, without resorting to the biochemical isolation of mitochondria and a subsequent HPLC analysis of glutathione content. Of note was the heterogeneity in MC1B fluorescence between neurons. The phase bright, small, bipolar neurons exhibited bright fluorescence, whereas, the large multipolar neurons were only weakly fluorescent. Differences in neuronal glutathione levels may underlie the differential vulnerability of neuronal populations in the spinal cord. This observation was consistent with the implication that the large multipolar neurons are more likely to die under oxidative stress as has been suggested for the greater susceptibility of motor neurons to excitotoxic insults (Urushitani, et al. (2000) J. Neurosci. Res. 61:443-448; Carriedo, et al. (2000) J. Neurosci. 20:240-250). A systematic correlation between neuronal morphology (aided by motor neuron-specific markers) and glutathione level was used to confirm this. A similar heterogeneity in MC1B fluorescence (and hence cellular glutathione levels) among glia cells has been reported (Chatterjee, et al. (1999) Glia 27:152-161).

### EXAMPLE 7

### Inhibition of Reactive Oxygen Species in Vitro

The ability of cysteine choline ester, N-acetyl cysteine choline ester, mitochondrial-targeted glutathione choline ester (Mito GSH), and mitochondrial-targeted N-acetyl-L-cysteine choline ester to prevent the depolarization of mitochondrial membrane potential induced by oxidative stress was assessed. Mitochondrial membrane potential was measured by a TPP+ (tetraphenyl phosphonium)-sensitive electrode. Rat heart mitochondria (1 mg protein/100 μl) were transferred to a beaker containing 0.9 ml of 150 mM KCl, 5 mM HEPES, 6 μM TPP+ and 5 mM succinate buffer. This caused a downward shift in the TPP+ signal due to a decrease in TPP+ concentration in the extramitochondrial solution as the probe was taken up by mitochondria. Approximately 1 minute later, mitochondria were subjected to oxidative stress by adding 5 μM rotenone (Complex I inhibitor) and 100 μM tert-butylhydroperoxide (t-BuOOH) to the buffer. This led to mitochondrial depolarization, resulting in release of intramitochondrial TPP+ as observed by an increase in TPP+ signal. Pretreatment of mitochondria with anti-oxidants (5 mM at 4° C. for 30 minutes, then resuspended mitochondria in drug free solution), prevented the oxidative stress-induced depolarization significantly.

### EXAMPLE 8

### Selective Delivery of N-Acetyl-L-Cysteine Improves Post-Ischemic Recovery in Rat Heart

The ability of mitochondrial-targeted N-acetyl-L-cysteine choline ester to improve post-ischemic recovery in rat heart was assessed. Male Sprague-Dawley rat hearts were retrograde (Langendorff) perfused with oxygenated Krebs Henseleit (KH) buffer in constant flow mode (12 mL/min/gram wet weight). Hearts were not electrically stimulated, and beat spontaneously at approximately 280 beats per minute. Left-ventricular pressure (LVP) was measured by a balloon inserted in the left ventricle, linked to a pressure transducer with digital recording at 500 Hz. Following an equilibration period of approximately 25 minutes, global normothermic ischemia was imposed for 25 minutes, followed by reperfusion for 30 minutes. For N-acetyl-L-cysteine treatment, the drug was dissolved in KH buffer and infused via a port just above the aortic perfusion canula, at a final concentration of 50 μM, for 10 minutes prior to the onset of ischemia.

Overall recovery of left-ventricular developed pressure (systolic minus diastolic) was 4.1% for control, and 15.7% for N-acetyl-L-cysteine treated hearts. It was also apparent that N-acetyl-L-cysteine appeared to delay the onset of ischemic contracture.

### EXAMPLE 9

### Prevention of Mitochondrial Membrane Potential Depolarization Induced by Oxidative Stress

The ability of cysteine choline ester (CYS CE), N-acetyl cysteine choline ester (NAC CE), glutathione choline ester (Mito GSH), and S,N-acetyl-L-cysteine choline ester (Mito NAC) to prevent the depolarization of mitochondrial membrane potential induced by oxidative stress was assessed (FIG. 2). Mitochondrial membrane potential was measured by safranine. Safranine is a positively charged dye that accumulates in mitochondria on establishment of an electrical potential across the mitochondrial inner membrane. However, its fluorescence is quenched by its accumulation in mitochondria in response to mitochondrial membrane potential. Rat heart mitochondria (0.2 mg protein/200 μl) were transferred to a well containing 0.3 ml of 150 mM KCl, 5 mM HEPES, 15, μM safranine, 5 mM succinate buffer, 5 μM rotenone and 100 μM t-BuOOH. The mitochondria were placed in a multiplate reader.

Fluorescence measurements were made with excitation and emission wavelengths of 485 and 585 nm, respectively. Rotenone and t-BuOOH induced mitochondrial membrane potential resulting in a release of intramitochondrial safranine indicated by an increase in fluorescence signal. Pretreatment of mitochondria with antioxidants selectively delivered to the mitochondria, glutathione choline ester (Mito GSH), N-acetyl cysteine choline ester, N,S-acetyl cysteine choline ester (Mito NAC), and cysteine choline ester (5 mM for 30 minutes and then resuspended in a drug free solution) diminished the release of intramitochondrial safaranine, indicating a protective effect of these compounds.

### EXAMPLE 10

### Delay of Oxidative Stress-Induced Depolarization of Mitochondrial Membrane Potential

Using tetramethylrhodamine methyl ester (TMRE) as an indicator of mitochondrial membrane potential, the ability of mitochondrial-targeted glutathione choline ester (Mito GSH) to delay the onset of H₂0₂-induced depolarization of mitochondria membranes was assessed (FIG. 3). TMRE is a lipophilic cation that partitions selectively into the negatively charged mitochondria. Neonatal cultured myocytes (6 days in culture) were loaded with 10 nM TMRE for 60 minutes at 37° C. The myocytes were pretreated with either 50 μM or 100 μM Mito GSH or 100 μM non-targeted glutathione for 30-minutes and then washed to remove the antioxidants from the solution in which myocytes were suspended. As a control, myocytes were not pretreated with any drug. TMRE was excited at 555 nm and fluorescence emission was detected at 590 nm. Fluorescence images were taken every 2 minutes. At the arrow (FIG. 3), myocytes were subjected to oxidative stress by adding 50 μM H₂0₂. Plots were normalized to baseline, and are shown as F/F0, where F is the emitted fluorescence at any given time and F0 is the baseline fluorescence before addition of H₂0₂. Mito GSH pretreatment delayed onset of H₂0₂-induced depolarization and loss of TMRE fluorescence. The traces were drawn from the mean values of 7-10 experiments.

Time-lapse traces of TMRE fluorescence from cardiac myocytes after H₂0₂ treatment in control and glutathione choline ester (Mito GSH) pre-treated cells were obtained. TMRE was used as an indicator of mitochondrial membrane potential. The myocytes were pretreated with 50 μM Mito GSH for 30 minutes and then washed to remove the antioxidant from the solution in which myocytes were suspended. In control, myocytes were not pretreated with any drug. TMRE was excited at 555 run and fluorescence emission was detected at 590 nm. Fluorescence images were taken every 2 minutes to avoid photobleaching and phototoxicity. Myocytes were subjected to oxidative stress by adding 50 μM H₂0₂. In the control cell, the TMRE fluorescence was completely invisible 32 minutes after H₂0₂ treatment. However, in Mito GSH-pretreated cells, the TMRE fluorescence persisted for 50 minutes. This experiment demonstrated that Mito GSH pretreatment delayed H₂0₂-induced mitochondrial membrane depolarization.

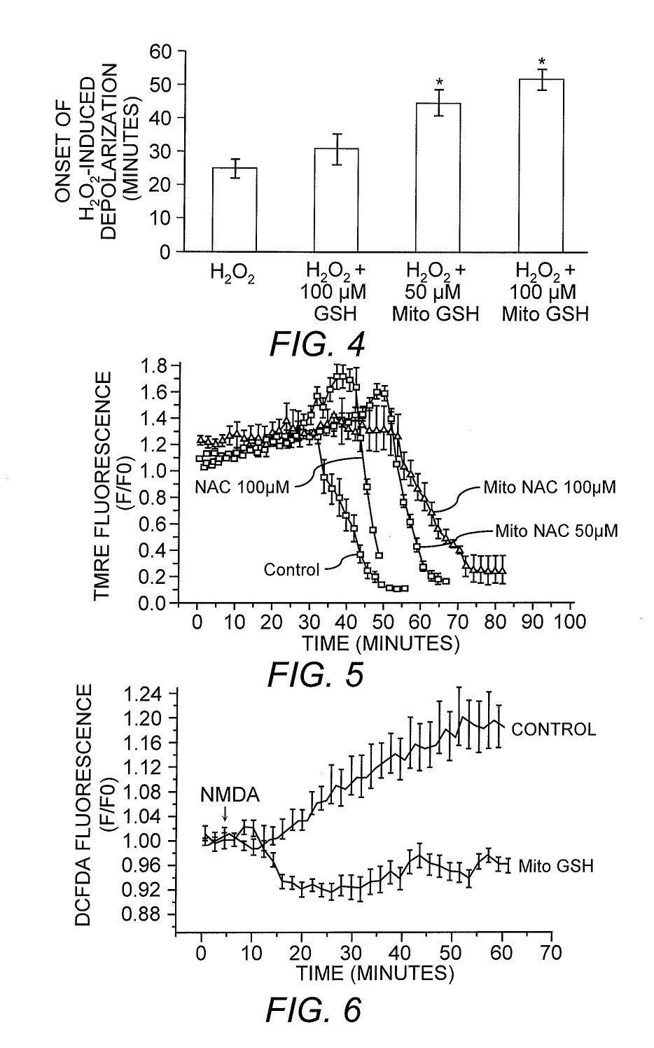

The latency of H₂0₂-induced depolarization of mitochondrial membrane potential in control (H₂0₂), glutathione (GSH), and glutathione choline ester (Mito GSH) is represented in FIG. 4. As shown, GSH (100 μM) did not significantly increase the time for onset of H₂0₂-induced depolarization. However, Mito GSH (50 and 100 μM) significantly enhanced the time for onset of H₂0₂-induced depolarization. Time for onset of H₂0₂-induced depolarization in Mito GSH (100 μM) pretreated myocytes was 53±3.6 minutes compared to 25±3.2 minutes for control myocytes (*p<0.05).

The ability of mitochondria-targeted antioxidant N-acetyl-L-cysteine choline ester (mito NAC) to delay the onset of H₂0₂-induced depolarization of mitochondrial membrane potential in cultured neonatal rat ventricular myocytes was assessed (FIG. 5). Using identical conditions and treatment concentrations as presented in the example showing that Mito GSH delays the onset of H₂0₂-induced depolarization, myocytes pretreated with Mito NAC showed a delayed onset of H₂0₂-induced depolarization and loss of fluorescence indicating a protective effect of this compound.

### EXAMPLE 11

### Protection Against N-Methyl-D-Aspartate-Induced ROS Generation in Brain Striatal Neurons

Intracellular reactive oxygen species (ROS) were measured by using the redox-sensitive dye, dichlorohydrofluorescein (H₂DCFDA). The thiol-reactive chloromethyl group binds to cellular thiols trapping the dye inside the cell where oxidation converts it to the fluorescent form, dichlorofluorescein (DCF). Cultured striatal neurons (10 days in culture) were loaded with 50 nM H₂DCFDA for 25 minutes. The neurons were excited at 488 nm and the image was acquired at 515 nm wavelength. ROS production was induced by treating the neurons with 100 μM N-methyl-D-aspartate (NMDA). An increase in DCFDA fluorescence by NMDA treatment reflected an increased production of ROS or oxidative stress. Pretreatment of neurons with 100 μM Mito GSH protected the neurons from ROS production (FIG. 6). Plots were normalized to baseline, and are shown as F/F0, where F is the emitted fluorescence at any given time and F0 is the baseline fluorescence before addition of NMDA. Mito GSH pretreatment prevented NMDA-induced increase in ROS production. The traces were drawn from the mean values of three experiments.

The effects of mitochondrial-targeted antioxidants upon onset of NMDA (100 μM) induced depolarization of mitochondrial membrane of brain striatal neurons are summarized in Table 1.

TABLE 1

| | Time of Onset of |
|---|---|
| Antioxidant Pretreatment | Depolarization (Minutes) |
| None (control) | 8.1 ± 1.4 |
| Glutathione | 9.2 ± 2.6 |
| Mito GSH | 17.5 ± 2.1* |
| N-acetyl cysteine | 10.5 ± 1.9 |
| Mito NAC | 20.5 ± 3.6* |

*p < 0.05

Tetramethylrhodamine methyl ester (TMRE) was used as an indicator of mitochondrial membrane potential. The neurons were pretreated with either 50 μm glutathione (GSH) or glutathione choline ester (Mito GSH) or N-acetyl-L-cysteine (NAC) or N-acetyl-L-cysteine choline ester (Mito NAC) for 30 minutes and then washed to remove the antioxidants from the solution in which neurons were suspended. Control neurons were not pretreated with any drug.

### EXAMPLE 12

### Inhibition of Ischemia-Induced Neurological Damage

Compounds of the present invention are administered to rats to assess their ability to attenuate ischemia/reperfusion injury to brain tissue caused by a focal cerebral ischemia model. Focal cerebral ischemia (45 minutes) is induced in anesthetized rats using standard procedures (i.e., occluding the middle cerebral artery (MCA) with an intra-luminal suture through the internal carotid artery). Buffered solutions containing the compounds of the present invention are administered pre-ischemia and post-ischemia to assess their efficacy. The rats are scored post-reperfusion for neurological deficits and then sacrificed after 24 hours of reperfusion. Infarct volume in the brain is assessed by 2,3,5-triphenyl tetrazolium chloride (TTC). Brain sections are immunostained for tumor necrosis factor (TNF-alpha) and inducible nitric oxide synthase (iNOS). It is expected that rats treated with compounds of the present invention will show a reduction in brain infarct volume and more favorable neurological evaluation score as compared to the untreated animals, which would be consistent with the in vitro results reports in preceding examples.

What is claimed is:

1. An amino acid-based antioxidant compound selectively delivered into the mitochondria of a cell.

2. A pharmaceutical composition comprising the antioxidant of claim 1 in admixture with a pharmaceutically acceptable carrier.

3. A method for producing a compound of claim 1 comprising linking an amino acid-based antioxidant to a delivery moiety which selectively delivers the antioxidant to the mitochondria of a cell.

4. A method of inhibiting oxidative stress-induced cell injury or death comprising contacting a cell with the compound of claim 1, whereby the compound is taken up by the cell and is selectively delivered into the mitochondria of the cell, thereby scavenging oxidative free radicals or reactive oxygen species to inhibit oxidative stress-induced cell injury or death.

5. A method of treating a condition associated with oxidative stress-induced cell injury or death comprising administering an effective amount of the composition of claim 2 to a patient having a condition associated with oxidative stress-induced cell injury or death, whereby the compound is taken up by cells at risk of oxidative stress-induced injury or death, and is selectively delivered into the mitochondria of the cells to inhibit oxidative stress-induced injury or death thereof, thereby treating the condition.
